(19) United States
(12) Patent Application Publication
(10) Pub. No.: US 2024/0169822 A1
(43) Pub. Date: May 23, 2024

(54) SMART ACTION SYSTEM FOR SMART ACTION SELECTION AND INITIATION

(71) Applicant: ARRIS Enterprises LLC, Suwanee, GA (US)

(72) Inventors: Vinod JATTI, Bangalore (IN); Kanakendiran RAMACHANDIRAN, Chennai (IN)

(21) Appl. No.: 18/511,011

(22) Filed: Nov. 16, 2023

Related U.S. Application Data
(60) Provisional application No. 63/427,507, filed on Nov. 23, 2022.

(51) Int. Cl. G08B 21/24

(52) U.S. Cl. CPC G08B 21/24

(57) ABSTRACT

A smart action system can provide a user with an improved experience by monitoring for one or more environmental parameters. The one or more environmental parameters can be associated with a smart action. The smart action can cause one or more actions such as a selection of one or more notifications and/or any other action associated with a network device. The smart action can, for example, change an aspect of a network device, such as to adjust a volume control, provide a reminder to a user regarding a schedule, such as related to a medicine or other health condition, and/or any other action.

### BACKGROUND

Wireless in general, and Wi-Fi (wireless fidelity) in particular have become ubiquitous in networking environments such that many devices that previously relied on manual readouts and displays also provide the same information over wireless technologies. This is even more important as there is a concomitant availability of software applications that run on wireless devices (such as mobile phones) that can analyze data. For example, as healthcare costs continue to increase, there is an increasing desire with aging adults to stay in place (in home) for extended care services or even for convenience users are seeking assistance with health issues without having to travel to a medical provider or participate in a telemedicine call. However, many users may have difficult with traditional techniques for interfacing with a user. Thus, there is a need to provide an improved system for presenting useful or important information to a user.

### SUMMARY

Generally, there are many devices in the market that operate or behave as point solutions for providing medical diagnostic information associated with a health condition of a user. Each solution may have an associated device and an associated application that runs on the associated device. However, these solutions or technologies can include an interface that does not adequately provide a user with the required information. For example, a user can be an elderly person, a person with an impairment, such as a person with a hearing impairment, or any other user that experiences difficulty with traditional network devices. Such users may experience a poor quality of experience (QoE) with traditional systems. According to one or more aspects of the present disclosure, a smart action system can automatically and dynamically detect or identify one or more environmental parameters to select and initiate one or more smart actions.

An aspect of the present disclosure provides a smart action system for selecting a smart action. The smart action system comprises a memory storing one or more computer-readable instructions and a processor configured to execute the one or more computer-readable instructions to monitor for one or more environmental parameters, receive the one or more environmental parameters, identify at least one of the one or more environmental parameters as associated with a user, select the smart action based on the at least one of the one or more environmental parameters, and initiate the smart action.

In an aspect of the present disclosure, the processor is further configured to execute the one or more instructions to receive a user input based on the initiating the smart action.

In an aspect of the present disclosure, the notification is associated with a reminder.

In an aspect of the present disclosure, the initiating the smart action comprises providing a notification to the user.

In an aspect of the present disclosure, the notification instructs the user regarding a health condition.

In an aspect of the present disclosure, the one or more environmental parameters comprise a sound associated with any of a user, an animal, a sensing device, or any combination thereof.

In an aspect of the present disclosure, the one or more environmental parameters are received from a client device, a sensing device, or both.

An aspect of the present disclosure provides a smart action system to select a smart action. The method comprises monitoring for one or more environmental parameters, receiving the one or more environmental parameters, identifying at least one of the one or more environmental parameters as associated with a user, selecting a smart action based on the at least one of the one or more environmental parameters, and initiating the smart action.

In an aspect of the present disclosure, the method further comprises receiving a user input based on the initiating the smart action.

In an aspect of the present disclosure, the method is such that initiating the smart action comprises providing a notification to the user.

In an aspect of the present disclosure, the method is such that the notification is associated with a reminder.

In an aspect of the present disclosure, the method is such that the notification instructs the user regarding a health condition.

In an aspect of the present disclosure, the method is such that the one or more environmental parameters comprise a sound associated with any of a user, an animal, a sensing device, or any combination thereof.

In an aspect of the present disclosure, the one or more environmental parameters are received from a client device, a sensing device, or both.

An aspect of the present disclosure provides a non-transitory computer-readable medium of a smart action system storing one or more instructions for selecting a smart action, which when executed by a processor of the network device, cause the smart action system to perform one or more operations including the steps of the methods described above.

According to various aspects of the present disclosure, one or more novel solutions can provide a smart action system for selecting and initiating a smart action based on one or more received environmental parameters.

### BRIEF DESCRIPTION OF DRAWINGS

In the drawings, like reference numbers generally indicate identical, functionally similar, and/or structurally similar elements.

FIG. 1 is a schematic diagram of a network environment, according to one or more aspects of the present disclosure;

FIG. 2 is a more detailed block diagram illustrating various components of a network device, according to one or more aspects of the present disclosure;

FIG. 3 is an illustration of a smart action system in a network environment, according to one or more aspects of the present disclosure;

FIG. 4 is an illustration of providing a notification associated with a smart action to a user via an output device, according to one or more aspects of the present disclosure;

FIG. 5 illustrates exemplary aspects of environmental parameters associated with smart actions, according to one or more aspects of the present disclosure;

FIG. 6 is an exemplary network device, according to one or more aspects of the present disclosure;

FIG. 7 is a process for a smart action system to select one or more smart actions based on one or more environmental parameters, according to one or more aspects of the present disclosure; and

FIG. 8 is a flow chart illustrating a method for selecting one or more smart actions based on one or more environmental parameters, according to one or more aspects of the present disclosure.

### DETAILED DESCRIPTION

The following detailed description is made with reference to the accompanying drawings and is provided to assist in a comprehensive understanding of various example embodiments of the present disclosure. The following description includes various details to assist in that understanding, but these are to be regarded merely as examples and not for the purpose of limiting the present disclosure as defined by the appended claims and their equivalents. The words and phrases used in the following description are merely used to enable a clear and consistent understanding of the present disclosure. In addition, descriptions of well-known structures, functions, and configurations may have been omitted for clarity and conciseness. Those of ordinary skill in the art will recognize that various changes and modifications of the examples described herein can be made without departing from the spirit and scope of the present disclosure.

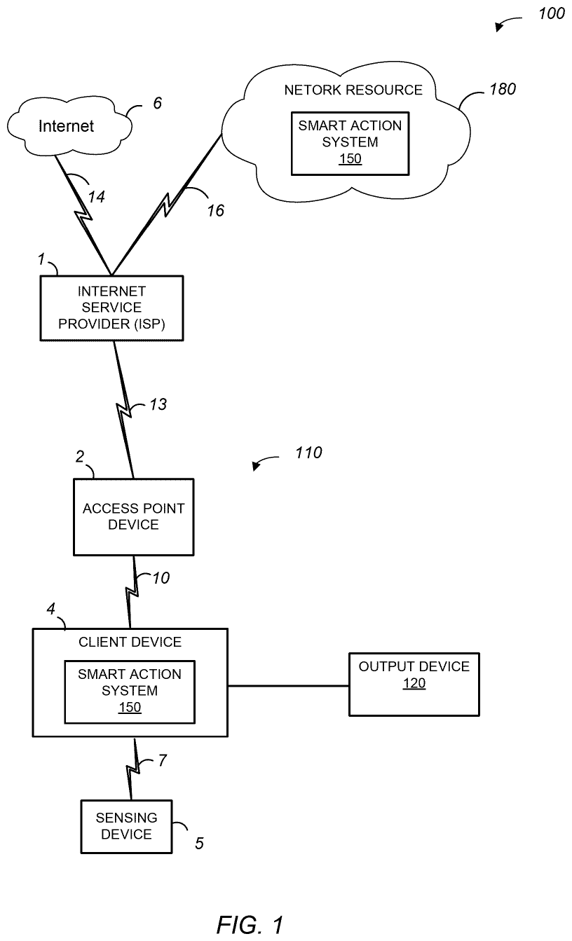

FIG. 1 is a schematic diagram of a network environment 100, according to one or more aspects of the present disclosure. For example, a network environment 100 can provide for aggregation of user data from a user, multiple network devices, and/or sources. An example network environment 100 can be related to a caregiving network for a user (a patient) such that one or more aspects associated with the user (for example, biometric data (such as from a third-party user-wearable device), an audio and/or visual interface, etc.) can be used to select and initiate a smart action.

It should be appreciated that various example embodiments of inventive concepts disclosed herein are not limited to specific numbers or combinations of devices, and there may be one or multiple of some of the aforementioned electronic apparatuses in the network environment, which may itself consist of multiple communication networks and various known or future developed wireless connectivity technologies, protocols, devices, and the like.

As shown in FIG. 1, the main elements of the network environment 100 include a network 110 comprising an access point device 2 connected to any of the Internet 6, a smart action system 150, a network resource 180, any other cloud storage and/or repository, or any combination thereof via an Internet Service Provider (ISP) 1 and also connected to different wireless devices or network devices such as one or more client devices 4 and one or more sensing devices 5. The network environment 100 shown in FIG. 1 includes wireless network devices (for example, one or more client devices 4) that may be connected in one or more wireless networks within the network environment 100. Additionally, there could be some overlap between wireless devices in the different networks. That is, one or more network or wireless devices could be located in more than one network.

The ISP 1 can be, for example, a content provider or any computer for connecting the access point device 2 to any of a network resource 180, the Internet 6, the network resource 180, the smart action system 150, or any combination thereof via the ISP 1. For example, Internet 6 can be a cloud-based service that provides access to a network resource 180, such as a cloud-based repository, accessible via ISP 1 where the network resource comprises information associated with a detected one or more environmental parameters. The network resource 180 can comprise any of a website, a database, a portal, any other repository for storing and retrieving information, or any combination thereof.

The smart action system 150 can provide monitoring, aggregation and/or controlling of data, such as one or more environmental parameters, associated with a user of network 110. The smart action system 150 can be part of or included within a network resource 180, any other network device (such as a cloud server or a client device 4) or any combination thereof. For example, the smart action system 150 can comprise and/or include an interface for accessing one or more databases or repositories of the network resource 180, any other network device, or both. The one or more databases can store information associated with an environmental parameter, such as any of data collected by one or more sensing devices 5, user profile information, medical information associated with a user, such as a medical history, one or more results from one or more queries, any other information that can be used in a subsequent session, any other information that can be used for a session associated with a similar environmental parameter, or any combination thereof. The smart action system 150 can select one or more smart actions associated with one or more environmental parameter and initiate the one or more selected smart actions. In one or more embodiments, the smart action system 150 can communicate with any one or more external repositories of Internet 6 via ISP 1 or internal repositories, such as a notification repository. In one or more embodiments, a sensing device can be directly or indirectly coupled to the smart action system 150, for example, sensing device 5 can be connected to smart action system 150 of client device 4 via a connection 7.

The connection 14 between the Internet 6 and the ISP 1, the connection 16 between the network resource 180 and the ISP 1, the connection 13 between the ISP 1 and the access point device 2, and any other connection 10 and/or 7 can be implemented using a wide area network (WAN), a virtual private network (VPN), metropolitan area networks (MANs), system area networks (SANs), a data over cable service interface specification (DOCSIS) network, a fiber optics network (for example, FTTH (fiber to the home) or FTTX (fiber to the x), or hybrid fiber-coaxial (HFC)), a digital subscriber line (DSL), a public switched data network (PSDN), a global Telex network, or a 2G, 3G, 4G, 5G, or 6G network, for example. Any of the connections 7, 10, 13, 14, and 16, or any combination thereof (collectively referred to as network connections or connections) can further include as some portion thereof a broadband mobile phone network connection, an optical network connection, or other similar connections.

The access point device 2 can be, for example, an access point and/or a hardware electronic device that may be a combination modem and gateway that combines the functions of a modem, an access point (AP), and/or a router for providing content received from the ISP 1 to one or more network devices (for example, one or more client devices 4) in the network environment 100, or any combination thereof. It is also contemplated by the present disclosure that the access point device 2 can include the function of, but is not limited to, a universal plug and play (UPnP) simple network management protocol (SNMP), an Internet Protocol/Quadrature Amplitude Modulator (IP/QAM) set-top box (STB) or smart media device (SMD) that is capable of decoding audio/video content, and playing over-the-top (OTT) or multiple system operator (MSO) provided content. The access point device 2 may also be referred to as a residential gateway, a home network gateway, or a wireless access point (AP).

The client devices 4 can be, for example, hand-held computing devices, personal computers, electronic tablets, mobile phones, smart phones, smart speakers, Internet-of-Things (IoT) devices, iControl devices, portable music players with smart capabilities capable of connecting to the Internet, cellular networks, and interconnecting with other devices via Wi-Fi and Bluetooth, or other wireless hand-held consumer electronic devices capable of executing and displaying content received through the access point device 2. Additionally, the client devices 4 can be a television (TV), a smart media device, an IP/QAM set-top box (STB) or a streaming media decoder (SMD) that is capable of decoding audio/video content, and playing over OTT or MSO provided content received through the access point device 2. In one or more embodiments, the client device 4 can comprise any network device associated with a user for interacting with any type of one or more sensing devices 5. For example, the client device 4 can interact with a plurality of sensing devices 5 where each sensing device 5 senses one or more aspects associated with a user or an environment, such as a health condition. In one or more embodiments, one or more sensing devices 5 are included within or local to (built-in) the client device 4. In one or more embodiments, the client device 4 can be a network device that includes a smart action system 150 or is part of a smart action system 150. In one or more embodiments, the client device 4 is directly or indirectly coupled to a output device 120 for displaying, providing or otherwise presenting one or more notifications associated with one or more smart actions selected by the smart action system 150 based on one or more environmental parameters. The output device 120 can comprise any of a monitor, a television, an audio and/or video (A/V) system, any other device capable of providing playback of audio, video, and/or text, or any combination thereof.

One or more sensing devices 5 can connect to a client device 4, for example, via a connection 7. The one or more sensing devices 5 can comprise one or more health diagnostic devices, such as any of an optical instrument (such as a camera, an image capture device, or any other visual user interface device, any device for capturing an image, a video, a multi-media video, or any other type of data, or a combination thereof), a biometric sensor, a biometric tracker or sensor, ambient temperature sensor (such as a thermometer), a light sensor, a humidity sensor, a motion detector (such as, an infrared motion sensor or Wi-Fi motion sensor), a facial recognition system, a medical diagnostic sensor (such as, a pulse oximeter or any other oxygen saturation sensing system, a blood pressure monitor, a temperature sensor, a glucose monitor, etc.), a voice recognition system, a microphone (such as, a far field voice (FFV) microphone) or other voice capture system, any other sensing device, or a combination thereof.

It is contemplated by the present disclosure that the smart action system 150, the access point device 2, and the client device 4 include electronic components or electronic computing devices operable to receive, transmit, process, store, and/or manage data and information associated with the network environment 100, which encompasses any suitable processing device adapted to perform computing tasks consistent with the execution of computer-readable instructions stored in a memory or a computer-readable recording medium (for example, a non-transitory computer-readable medium). Further, any, all, or some of the computing components in the smart action system 150, access point device 2, and the client device 4 may be adapted to execute any operating system, including Linux, UNIX, Windows, MacOS, DOS, and Chrome OS as well as virtual machines adapted to virtualize execution of a particular operating system, including customized and proprietary operating systems. The smart action system 150, the access point device 2, and the client device 4 are further equipped with components to facilitate communication with other computing devices or network devices over the one or more network connections to local and wide area networks, wireless and wired networks, public and private networks, and any other communication network enabling communication in the network environment 100. As illustrated in FIG. 6, any one or more devices of network environment 100, such as a client device 4, can comprise a network device 200.

FIG. 6 illustrates a network device 200, according to one or more aspects of the present disclosure. The network device 200 can include an optical instrument or image capture device (such as a camera 152 or any other device that can obtain one or more visuals of a user), an audio input device 154 (such as a microphone, a microphone array, a far field voice (FFV) solution, a voice assistant, any other voice interface, etc.), an audio output device (such as a speaker 156), a sensing device 5, an interactive interface 610 (such as a user interface and/or an interface to an output device 120), and a smart device system 150. In one or more embodiments, the smart device system 150 is connected to locally or remote from (for example, a network resource 180) the network device 200. In one or more embodiments, any one or more components of the network device 200 can be included within or connected to the network device 200. The network device 200 can include any of one or more ports or receivers, for example, a Wi-Fi (such as a Wi-Fi5 (dual-band simultaneous (DBS)) port 158, a BLE port 160, an LTE port 162, an infrared (IR) blaster port 164, and IR receiver port (166), an Ethernet port 168, an HDMI-Out port 170, an HDMI-In port 172, an external power supply (such as a universal serial bus type-C (USB-C)), an LED output 176, or any combination thereof. The sensing device 5 can include any one or more types of sensors associated with medical diagnostics such as any of a power sensor, a temperature sensor, a light sensor, a humidity sensor, a motion sensor, a biometric sensor (such as a blood pressure monitor, oxygen saturation meter, pulse meter, etc.), any other type of sensor, or any combination thereof. In one or more embodiments, the sensing device 5 can be an IoT device.

The network device 200 can utilize one or more artificial intelligence technologies to analyze data associated with a user (also referred to as user data) and provide the user with information based on the data. The network device 200 can receive data, such as one or more environmental parameters, associated with the user from any of one or more sensor devices 5 (such as a health diagnostic or biometric device), an optical instrument, an audio input device, the user interface, such as a keyboard, a mouse, a remote control, a touchscreen, a BLE device, any other user interface device, or any combination thereof), any other input, or any combination thereof. As an example, the network device 200 can transmit a request to the user via an audio output device and receive a response as data via any of the audio input device, the camera, any one or more sensor devices 5, or any combination thereof. In addition, the network device 200 can retrieve data, for example, one or more environmental parameters, associated with the user from a network or cloud resource 180, such as from the Internet 6, a repository (for example, a diagnostic portal system 150), or both. All the data received can be aggregated and analyzed by the network device 200, the diagnostic portal system 150 or both to provide the user with any of a solution, such as a diagnosis, a request for additional data, or both. The network device 200 can provide a notification to a user via the interactive interface 610, an output device 120, or both associated with one or more smart actions selected by the smart action system 150 based on one or more environmental parameters. The network device 200 can access and display the notification associated with a user based on a profile configuration 250 associated with a user identifier 260 of the user. In one or more embodiments, the smart action system 150, the network device 200, or both can utilize data, such as one or more environmental parameters, associated with a user to select a corresponding smart action.

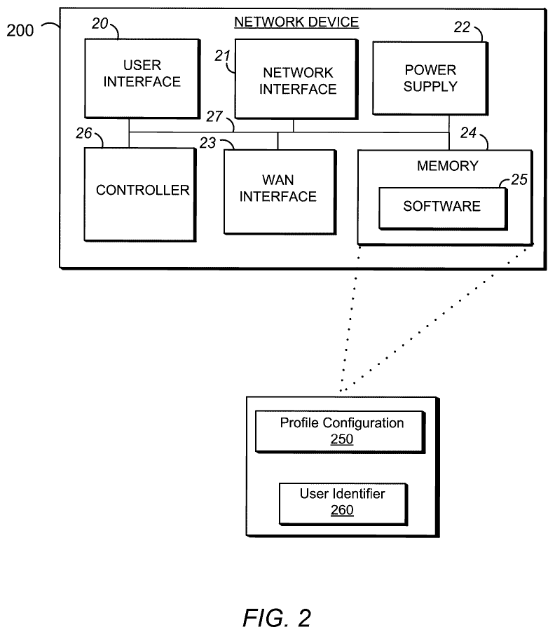

FIG. 2 is a more detailed block diagram illustrating various components of an exemplary network device 200, such as a network device comprising a smart action system 150, an access point device 2, a client device 4, any other network device, or any combination thereof implemented in, for example, a network 110 of FIG. 1 and/or a network resource 180 of a network environment 100, according to one or more aspects of the present disclosure.

The network device 200 can be, for example, a computer, a server, any other computer device with smart capabilities capable of connecting to the Internet, cellular networks, and interconnecting with other network devices via Wi-Fi and Bluetooth, or other wireless hand-held consumer electronic device capable of interacting with a user and/or requesting and/or analyzing user data, for example, using a smart action system 150, according to one or more aspects of the present disclosure. The network device 200 includes one or more internal components, such as a user interface 20, a network interface 21, a power supply 22, a controller 26, a WAN interface 23, a memory 34, and a bus 27 interconnecting the one or more elements.

The power supply 22 supplies power to the one or more internal components of the network device 200 through the internal bus 27. The power supply 22 can be a self-contained power source such as a battery pack with an interface to be powered through an electrical charger connected to an outlet (e.g., either directly or by way of another device). The power supply 22 can also include a rechargeable battery that can be detached allowing for replacement such as a nickel-cadmium (NiCd), nickel metal hydride (NiMH), a lithium-ion (Li-ion), or a lithium Polymer (Li-pol) battery.

The user interface 20 includes, but is not limited to, push buttons, a keyboard, a keypad, a liquid crystal display (LCD), a thin film transistor (TFT), a light-emitting diode (LED), a high definition (HD) or other similar display device including a display device having touch screen capabilities so as to allow interaction between a user and the network device 200, for example, for a user to enter any one or more profile configurations 250, a user identifier 260, any other user data associated with a user or network device, or a combination thereof that are stored in memory 34. The network interface 20 can include, but is not limited to, various network cards, interfaces, and circuitry implemented in software and/or hardware to enable communications with and/or between the smart action system 150, the access point device 2, and/or a client device 4 using any one or more of the communication protocols in accordance with any one or more connections (for example, as described with reference to FIG. 1). In one or more embodiments, the user interface 20 enables communications with a sensing device 5, directly or indirectly.

The memory 24 includes a single memory or one or more memories or memory locations that include, but are not limited to, a random access memory (RAM), a dynamic random access memory (DRAM) a memory buffer, a hard drive, a database, an erasable programmable read only memory (EPROM), an electrically erasable programmable read only memory (EEPROM), a read only memory (ROM), a flash memory, logic blocks of a field programmable gate array (FPGA), an optical storage system, a hard disk or any other various layers of memory hierarchy. The memory 24 can be used to store any type of instructions, software, or algorithms including software 25, for example, a smart action system 150, for controlling the general function and operations of the network device 200 in accordance with one or more embodiments. In one or more embodiments, memory 24 can store any one or more profile configurations 250 associated with one or more user identifiers 260 so as to provide (for example, by a smart action system 150) selection and initiation of a smart action, such as based on one or more environmental parameters from one or more sensing devices 5. The one or more user identifiers 260 can comprise a unique identifier associated with one or more users, one or more network devices, or both. The one or more user identifiers 260 can be associated with one or more profile configurations 250 which include information associated with a user. In one or more embodiments, the profile configuration 250 and/or the user identifier 260 is stored in any type of storage medium local to or remote from the network device 200.

The controller 26 controls the general operations of the network device 200 and includes, but is not limited to, a central processing unit (CPU), a hardware microprocessor, a hardware processor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software including the software 25 which can include one or more computer-readable instructions for a smart action system 150 in accordance with one or more embodiments. Communication between the components (for example, 20-26) of the network device 200 may be established using an internal bus 27.

The network interface 21 can include various network cards, interfaces, and circuitry implemented in software and/or hardware to enable communications with any one or more other network devices, for example, any of a client device 4, ISP 1, any other network device, or a combination thereof. The communications can utilize an interactive assistant connection that allows for an interface between the network device 200 and a user. The network interface 21 can include multiple radios or sets of radios (for example, a 2.4 GHz radio, one or more 5 GHz radios, and/or a 6 GHz radio), which may also be referred to as wireless local area network (WLAN) interfaces.

The wide area network (WAN) interface 23 may include various network cards, and circuitry implemented in software and/or hardware to enable communications between the access point device 2 and the ISP 1 using the wired and/or wireless protocols in accordance with connection 13 (for example, as described with reference to FIG. 1).

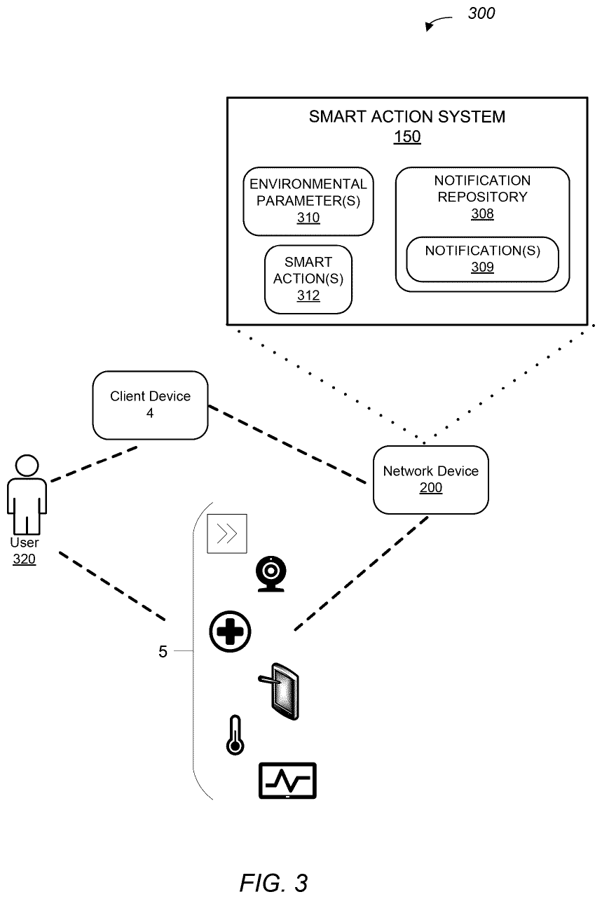

FIG. 3 illustrates a smart action system 150 of a network device 200 in a network environment 300, according to one or more aspects of the present disclosure. The network environment 300 can be the same as similar to network environment 150 of FIG. 1. The network environment 300 provides a network for analysis of one or more environmental parameters so as to select and/or initiate a smart action. The network environment 300 includes one or more client devices 4, a network device 200, and one or more sensing devices 5 (for example, a health diagnostic and/or biometric device). In one or more embodiments, the network device 200 and/or one or more other devices provides an interface to one or more sensing devices 5. While FIG. 3 illustrates a network environment 300 with a network device 200, the present disclosure contemplates any type of network environment 300 that includes a smart action system 150.

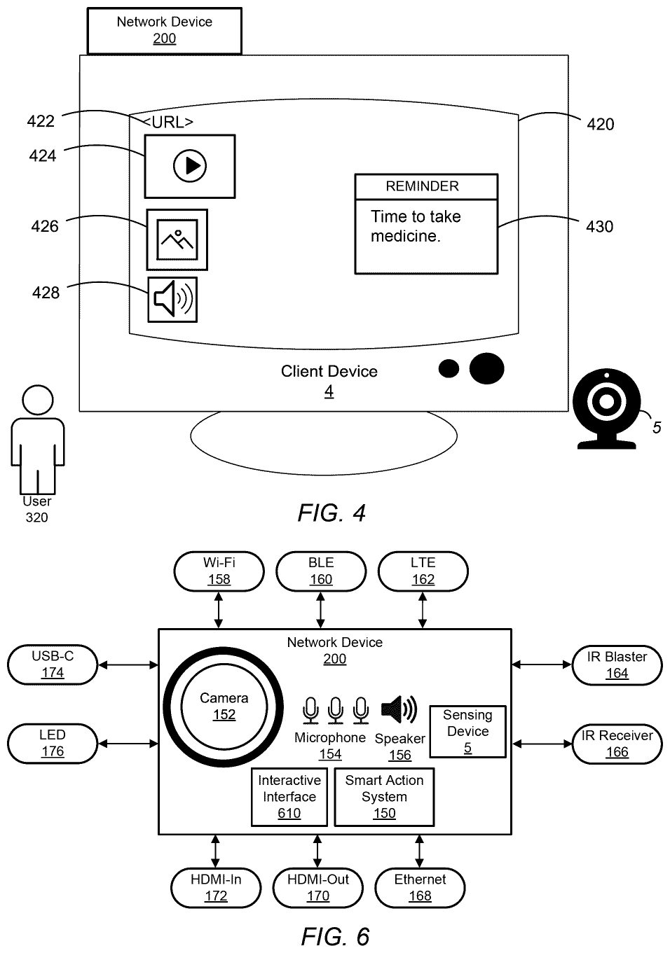

The smart action system 150 can provide diagnostic analysis, management, control, and access of user data, for example, for one or more environmental parameters 310 associated with one or more users 320 so as to select and initiate a smart action. The data or the one or more environmental parameters 310 can be stored as part of the smart action system 150, another network resource 180, and/or another network device 200. The smart action system 150 can comprise one or more smart actions 312, one or more environmental parameters 310, and a notification repository 308. The one or more smart actions 312 can be indicative of one or more actions, such as any of providing or sending a notification (for example, a notification 309 of the notification repository 308, such as a notification 309 associated with a schedule and/or reminder), altering, changing or otherwise adjusting a device in the network (such as another network device, a client device 4, a sensing device 5, any other device, or any combination thereof), any other action, or any combination thereof. The one or more environmental parameters 310 can comprise a sound (such as associated with any of one or more users, an animal, and/or sensing device (for example, any of a smoke detector, a blood pressure monitor, any other biomedical/biometric and/or environmental device, or any combination thereof), a voice command, a voice response, an utterance (such as a keyword, a safe word, a wake word, etc.), an output from a sensing device 5 (such as an audio output, a video output, and/or data output), any other identifiable sound, audio, and/or video, or any combination thereof. A notification repository 308 can comprise one or more notifications 309 associated with one or more smart actions so as to provide information to a user 320. The notification repository 308 can comprise any type of non-transitory computer-readable storage medium as discussed with reference to FIG. 2. The notification repository 308 can store information for display to a user 320, for example, as discussed with reference to FIG. 4. The one or more notifications 309 of the notification repository 308 can be stored in any type of storage system including, but not limited to, a flat file system, a database, a table, a data structure, a data object, any other type of storage system, or any combination thereof. In one or more embodiments, the notification repository 308 is remote from the smart action system 150.

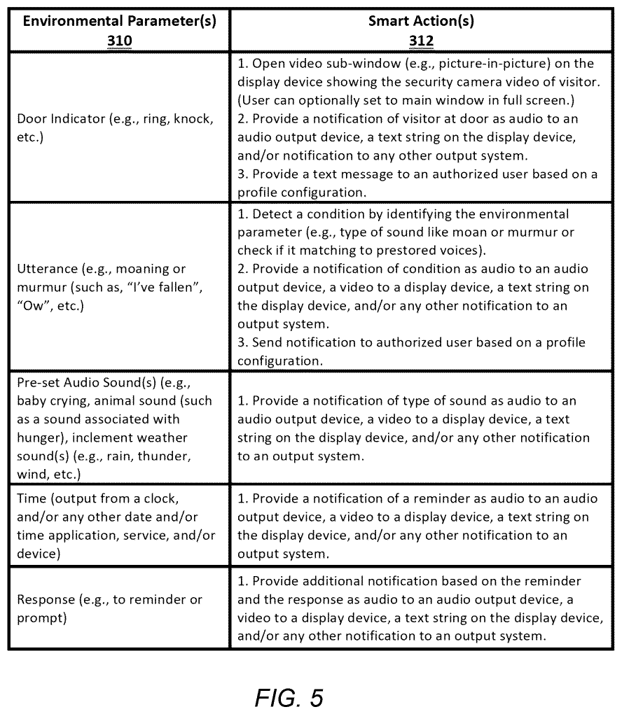

As an example, a smart action system 150 can receive one or more environmental parameters 310 from a client device, a sensing device 5, or any other source and select one or more smart actions based on the one or more environmental parameters 310, for example, as illustrated in FIG. 5. FIG. 5 illustrates one or more environmental parameters 310 associated with one or more smart actions 312, according to one or more aspect of the present disclosure. For example, the one or more environmental parameters 312 can comprise any of a door or access indicator (such as any of a ring from a doorbell associated with the door, an alarm associated with an access (for example, a door, a window, a garage door, etc.), a knock, any other indicator, or any combination thereof), an utterance (such as any of a moaning, a murmur, a phrase (for example, “I've fallen”, “help me”, etc.), an onomatopoeia (such as “ow”, etc.), a word (such as “help”, etc.), any other audible sound or any combination thereof), a pre-set audio sound (such as any of a baby crying, an animal sound (such as a sound associated with hunger), inclement or otherwise weather sound (such as any of rain, thunder, wind, any other weather sound, or any combination thereof), a time (such as an output from a clock, and/or any other date and/or time application, service, and/or device), a response (such as a response to a reminder or a prompt), or any combination thereof.

The smart device system 150 can detect or identify the one or more environmental parameters 310 and can select one or more smart actions 312 based on the detected one or more environmental parameters. For example, the smart device system 150. For example, the one or more smart actions 312 selected can comprise any of altering, adjusting, or otherwise changing a device (such as a opening a video sub-window (for example, picture-in-picture) on a display device so as to provide video from a sensing device (such as a camera)), providing a notification of a door or access indicator (such as any of providing an audio to an audio output device, a test string on the display device, an output to any other output system, or any combination thereof), providing a messaging to an authorized user based on a profile configuration (such as a text message to a client device 4 (for example, a mobile phone) of an authorized user), detecting a condition associated with the one or more environmental parameters (for example, detecting a medical condition such as a user is pain by matching received audio (such as a moan) to stored audio), provides a notification of a condition as audio to an audio output device, a video to a display device, a text string on a display device, and/or any other notification to any other output system, providing a notification of a type of sound as audio to an audio output device, a video to a display device, a text string on the display device, and/or any other notification to an output device, providing a notification of a reminder as audio to an audio output device, a video to a display device, a text string on the display device, and/or any other notification to an output system, providing an additional notification based on the reminder and response from the user as audio to an audio output device, a video to a display device, a text string on the display device, and/or any other notification to an output system, providing a notification of any environmental parameter to an authorized user based on a profile configuration associated with the user of the smart action system 150, or any combination thereof. While FIG. 5 illustrates certain smart actions 312 based on one or more environmental parameters 310, the present disclosure contemplates any number of smart actions 312 being based on any one or more environmental parameters 310 such that any given environmental parameter 310 can be associated with one or more smart actions 312 and any one or more smart actions 312 can be selected based on any one or more environmental parameters 310.

FIG. 4 is an illustration of providing a notification associated with a smart action 312 to a user 320 via an output device, according to one or more aspects of the present disclosure. A network device 200 can be connected to an output device, such as a client device 4, that includes a user interface 20, such as a display device 420 for displaying a notification received from a network device 200 connected to the client device 4. In one or more embodiments, the network device 200 is connected to a sensing device 5 that comprises an interactive interface, such as a camera or a visual interface. The network device 200 can send to the display device 420 a notification based on one or more environmental parameters 310 received by the network device. For example, the notification can comprise any of a uniform resource locator (URL) 422, a video 424, an image 426, an audio file 428, a list or text string 430, or any combination thereof. For example, the notification can be for a reminder or a schedule, such as for taking a medicine and/or for any other health condition. The notification can be based on a personal record or information associated with the user 320, such as any of a medical record, an audio record, a video record, an image regarding, a text-based record (for example, a record associated with a consultation), any other record, or any combination thereof. As an example, the notification can provide a name of a medicine, a date and/or time for taking the medicine, a dosage amount, a subsequent dosage time, and/or any other information. As another example, the notification can instruct the user regarding a health condition, for example, to begin an exercise routine or other activity. In one or more embodiments, the user 320 can provide a user input so as to cause the notification to become full-screen, to relocate on the screen, or any other adjustment.

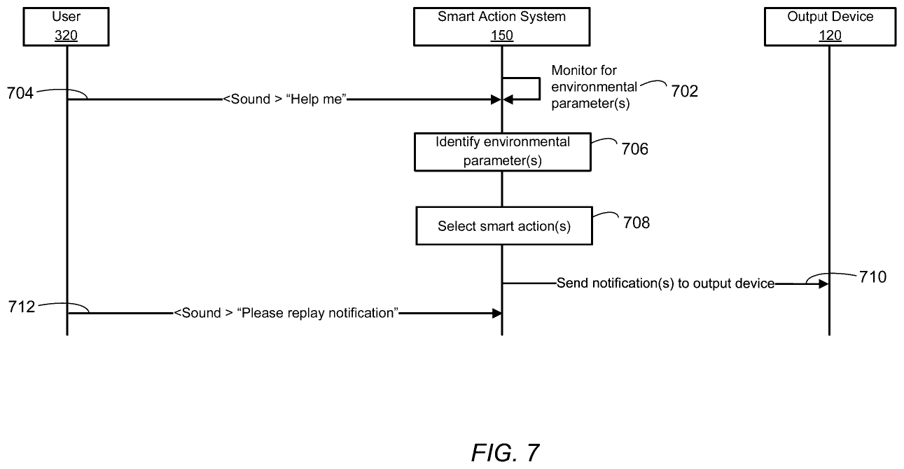

FIG. 7 is a process for a smart action system 150 to select one or more smart actions based on one or more environmental parameters, according to one or more aspects of the present disclosure. While FIG. 7 illustrates a smart action system 150, the present disclosure contemplates that a network device 200 can perform any one or more of the steps described with reference to FIG. 7. The network device 200 can comprise a smart action system 150, such as a client device 4, and may be programmed with one or more instructions that when executed by a processor controller causes the network device 200 to perform any one or more steps of FIG. 7. In FIG. 7, it is assumed that a network device 200 includes a respective controller and a respective software stored in a respective memory, as discussed herein in connection with any one or more figures which when executed by the respective controller performs the functions and operations in accordance with the example embodiments of the present disclosure (for example, including providing a notification to a user).

At step 702, a smart action system 150 of a network device 200 can monitor for one or more environmental parameters 310. For example, the network device 200 can be disposed or located at a premise associated with a user 320, such as a home or office. The one or more environmental parameters 310 can be predetermined and stored at the network device 200 and/or at a network resource 180. In one or more embodiments, the smart action system 150 is part of or within a network resource 180 and sends data to an access point device 2 and/or receives data from an access point device 2, for example, the data can comprise one or more environmental parameters 310. The one or more environmental parameters 310 can be indicative of a condition or issue associated with the user 320. For example, the one or more environmental parameters 310 can comprise any of a sound, a voice command, a voice response, an utterance, an output from a sensing device 5 (such as any of a video, an audio, biometric data, motion detection data, environment data (for example, temperature and/or humidity), any other sensing data, or any combination thereof), any other identifiable sound, audio, and/or video, or any combination thereof.

At step 704, the smart action system 150 can receive one or more environmental parameters, such as an audio from a user 320. For example, the smart action system 150 can receive from a user 320 a verbal message of “Help me” that is indicative of an issue associated with the user 320, such as the user needs assistance. In one or more embodiments, the one or more environmental parameters can be received directly from the user 320 as indicated in FIG. 7 or from a sensing device 5 associated with the user 320, for example, from an audio input device.

At step 706, the smart action system 150 can identify the one or more environmental parameters received at step 704. For example, the smart action system 150 can identify that the received one or more environmental parameters match a stored one or more environmental parameters. In one or more embodiments, identifying the one or more environmental parameters can comprise determining a user 320 and identifying which of the received one or more environmental parameters are associated with the user 320.

At step 708, the smart action system 150 can select a smart action 312 based on the one or more environmental parameters identified from step 706. In one or more embodiments, step 706 is omitted and at step 708 the smart action system 150 selects a smart action 312 based on the received one or more environmental parameters from step 704. For example, the selecting the one or more smart actions 312 can comprise the smart action system 150 determining that one or more environmental parameters are associated with one or more smart actions 312. The smart action system 150 can select all the one or more smart actions 312 determined or can select at least one of the one or more smart actions 312 determined based on the user 320 and/or any other criteria (such as any of a ranking of one or more smart actions 312 for a given one or more environmental parameters, a ranking of the one or more environmental parameters, any other basis, or any combination thereof).

In one or more embodiments, one or more smart actions 312 are associated with one or more notifications 309. At step 710, the smart action system 150 the smart action selected is a notification. The smart action system 150 sends or provides one or more notifications associated with the one or smart actions 312 to an output device 120 associated with the user 320. For example, the smart action system 150 can send a notification to any of a television, a tablet, a mobile phone, an A/V device, any other client device, or any combination thereof. In one or more embodiments, they smart action system 150 queries a notification repository 308 for the one or more notifications 309 based on the selected one or more smart actions 312. In one or more embodiments, the one or more notifications 309 are sent to an output device 120 associated with an authorized user as indicated by a profile configuration 250 associated with the user 320. In one or more embodiments, the one or more notifications can comprise any of a reminder, for example, a schedule, such as for a medicine that includes the medicine name, a dosage amount, etc., information associated with the one or more environmental parameters, the one or more smart actions, or any combination thereof.

In one or more embodiments, the one or more smart action 312 can cause an adjustment, alteration, change, or any other change in state of a network device within a network, such as indicated in FIG. 5. For example, the one or more smart actions 312 can cause a light to turn and/or off, a sensing device 5 to take a measurement and to send or report the measurement as one or more environmental parameters to the smart action system 150 and/or another network device, initiate a call with another user, such as an authorized user based on the profile configuration associated with the user 320, initiate an interactive session on a network device, such as a sub-window on a display device associated with an input/output device (such as a keyboard, a mouse, a camera, a speaker, a microphone, etc.), adjustment of a volume control, adjustment of a brightness control, adjustment of any other aspect of a display device, any other action associated with a smart action 312 associated with an environmental parameter, or any combination thereof.

At step 712, the smart action system 150 can receive a user input, for example, from a user 320, based on the one or more smart actions selected, such as the one or more notifications at step 710. For example, after initiating the smart action 312 the user 320 can provide a user input that requests that a notification be replayed or request that the volume or display be adjusted or changed. In one or more embodiments, if the smart action initiates a change to a network device, the user 320 can in response request that a verbal notification of the change be given, that a different action be taken, or that the action be repeated.

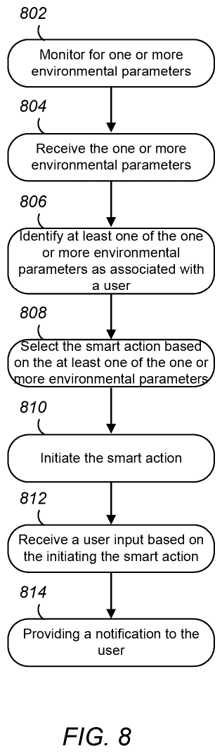

FIG. 8 illustrates a process for a smart action system 150 to provide a notification to a user, according to one or more aspects of the present disclosure. For the steps of FIG. 8, a network device 200 can comprise a smart action system 150, such as a client device 4, and may be programmed with one or more instructions that when executed by a processor controller causes the network device 200 to perform any one or more steps of FIG. 8. In FIG. 8, it is assumed that a network device 200 includes a respective controller and a respective software stored in a respective memory, as discussed herein in connection with any one or more figures which when executed by the respective controller performs the functions and operations in accordance with the example embodiments of the present disclosure (for example, including providing a notification to a user).

A smart action system 150 for selecting a smart action can comprise a memory storing one or more computer-readable instructions and a processor. The processor is configured to execute the one or more computer-readable instructions to perform one or more operations, such as the one or more operations of steps 802-814 of FIG. 8. At step 802, the smart action system 150 monitors for one or more environmental parameters. The monitoring can comprise parsing data received from any source via any interface of the smart action system 150, such as a sensing device 5. As indicated in step 804, the smart action system 150 can determine that one or more environmental have been received based on the parsing of this data.

At step 804, the smart action system 150 receives the one or more environmental parameters. The smart action system 150 can receive the one or more environmental parameters from one or more sources, such as any of a sensing device 5 that is included within or is coupled to the client device 4 that comprises the smart action system 150. The sensing device 5 can comprise any of a biometric sensing device, a camera, a microphone, any other sensing device, a user input via a user interface such as an interactive interface, or any combination thereof.

At step 806, the smart action system identifies at least one of the one or more environmental parameters as associated with a user. For example, the at least one of the one or more environmental parameters can be received from a client device, a sensing device, or both. According to one or more aspects of the present disclosure, the one or more environmental parameters comprise a sound associated with any of a user, an animal, a sensing device, or any combination thereof.

At step 808, the smart action system 150 selects the smart action based on the at least one of the one or more environmental parameters. At step 810, the smart action system 150 initiates the smart action, for example, as discussed with reference to FIG. 5. At step 812, the smart action system 150 receives a user input based on the initiating the smart action and at step 814, the smart action system 150 provides a notification to the user. The notification, according to one or more aspects of the present disclosure, instructs the user regarding a health condition.

While FIGS. 7 and 8 illustrate various steps of a method or process in a particular order, the present disclosure contemplates that any of the steps of FIGS. 7 and 8 can be performed in any order or repeatedly, and/or omitted (not performed). While the present disclosure discusses an aging-in-place environment, the present disclosure contemplates any other environment that requires remote monitoring be provided within a secure and private network environment such that on-demand and/or pre-authorized can be provided as well as an audit trail or log can be maintained. According to one or more example embodiments of inventive concepts disclosed herein, there are provided novel solutions for a smart action system to provide a notification to a user in a network based on one or more environmental parameters received by the smart action system.

Each of the elements of the present invention may be configured by implementing dedicated hardware or a software program on a memory controlling a processor to perform the functions of any of the components or combinations thereof. Any of the components may be implemented as a CPU or other processor reading and executing a software program from a recording medium such as a hard disk or a semiconductor memory, for example. The processes disclosed above constitute examples of algorithms that can be affected by software, applications (apps, or mobile apps), or computer programs. The software, applications, computer programs or algorithms can be stored on a non-transitory computer-readable medium for instructing a computer, such as a processor in an electronic apparatus, to execute the methods or algorithms described herein and shown in the drawing figures. The software and computer programs, which can also be referred to as programs, applications, components, or code, include machine instructions for a programmable processor, and can be implemented in a high-level procedural language, an object-oriented programming language, a functional programming language, a logical programming language, or an assembly language or machine language.

The term “non-transitory computer-readable medium” refers to any computer program product, apparatus or device, such as a magnetic disk, optical disk, solid-state storage device (SSD), memory, and programmable logic devices (PLDs), used to provide machine instructions or data to a programmable data processor, including a computer-readable medium that receives machine instructions as a computer-readable signal. By way of example, a computer-readable medium can comprise DRAM, RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Disk or disc, as used herein, includes compact disc (CD), laser disc, optical disc, digital versatile disc (DVD), floppy disk and Blu-ray disc. Combinations of the above are also included within the scope of computer-readable media.

The word “comprise” or a derivative thereof, when used in a claim, is used in a nonexclusive sense that is not intended to exclude the presence of other elements or steps in a claimed structure or method. As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise. Use of the phrases “capable of,” “configured to,” or “operable to” in one or more embodiments refers to some apparatus, logic, hardware, and/or element designed in such a way to enable use thereof in a specified manner.

While the principles of the inventive concepts have been described above in connection with specific devices, apparatuses, systems, algorithms, programs and/or methods, it is to be clearly understood that this description is made only by way of example and not as limitation. The above description illustrates various example embodiments along with examples of how aspects of particular embodiments may be implemented and are presented to illustrate the flexibility and advantages of particular embodiments as defined by the following claims, and should not be deemed to be the only embodiments. One of ordinary skill in the art will appreciate that based on the above disclosure and the following claims, other arrangements, embodiments, implementations and equivalents may be employed without departing from the scope hereof as defined by the claims. It is contemplated that the implementation of the components and functions of the present disclosure can be done with any newly arising technology that may replace any of the above-implemented technologies. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What we claim is:

1. A smart action system for selecting a smart action comprising:
a memory storing one or more computer-readable instructions; and
a processor configured to execute the one or more computer-readable instructions to:
monitor for one or more environmental parameters;
receive the one or more environmental parameters;
identify at least one of the one or more environmental parameters as associated with a user;
select the smart action based on the at least one of the one or more environmental parameters; and
initiate the smart action.

2. The network device of claim 1, wherein the processor is further configured to execute the one or more instructions to:
receive a user input based on the initiating the smart action.

3. The network device of claim 1, wherein initiating the smart action comprises:
providing a notification to the user.

4. The network device of claim 3, wherein the notification is associated with a reminder.

5. The network device of claim 3, wherein the notification instructs the user regarding a health condition.

6. The network device of claim 1, wherein the one or more environmental parameters comprise a sound associated with any of a user, an animal, a sensing device, or any combination thereof.

7. The network device of claim 1, wherein the one or more environmental parameters are received from a client device, a sensing device, or both.

8. A method for a smart action system to select a smart action, the method comprising:
monitoring for one or more environmental parameters;
receiving the one or more environmental parameters;
identifying at least one of the one or more environmental parameters as associated with a user;
selecting a smart action based on the at least one of the one or more environmental parameters; and
initiating the smart action.

9. The method of claim 8, further comprising:
receiving a user input based on the initiating the smart action.

10. The method of claim 8, wherein initiating the smart action comprises:
providing a notification to the user.

11. The method of claim 10, wherein the notification is associated with a reminder.

12. The method of claim 10, wherein the notification instructs the user regarding a health condition.

13. The method of claim 8, wherein the one or more environmental parameters comprise a sound associated with any of a user, an animal, a sensing device, or any combination thereof.

14. The method of claim 8, wherein the one or more environmental parameters are received from a client device, a sensing device, or both.

15. A non-transitory computer-readable medium of a smart action system storing one or more instructions for selecting a smart action, which when executed by a processor of the smart action system, cause the smart action system to perform one or more operations comprising:
monitoring for one or more environmental parameters;
receiving the one or more environmental parameters;
identifying at least one of the one or more environmental parameters as associated with a user;
selecting a smart action based on the at least one of the one or more environmental parameters; and
initiating the smart action.

16. The non-transitory computer-readable medium of claim 15, wherein the one or more instructions when executed by the processor further cause the smart action system to further perform the one or more operations comprising:
receiving a user input based on the initiating the smart action.

17. The non-transitory computer-readable medium of claim 15, wherein initiating the smart action comprises:
providing a notification to the user.

18. The non-transitory computer-readable medium of claim 17, wherein at least one of:
the notification is associated with a reminder; and
wherein the notification instructs the user regarding a health condition.

19. The non-transitory computer-readable medium of claim 15, wherein the one or more environmental parameters comprise a sound associated with any of a user, an animal, a sensing device, or any combination thereof.

20. The non-transitory computer-readable medium of claim 15, wherein the one or more environmental parameters are received from a client device, a sensing device, or both.
